(19) United States
(12) Patent Application Publication
(10) Pub. No.: US 2017/0311868 A1
(43) Pub. Date: Nov. 2, 2017

(54) System and Method for Screening Traumatic Head Injury

(71) Applicant: Sacred Brains, LLC, Montebello, NY (US)

(72) Inventors: Darron Bacal, Orange, CT (US); Scott Einiger, Montebello, NY (US)

(21) Appl. No.: 15/581,557

(22) Filed: Apr. 28, 2017

Related U.S. Application Data
(60) Provisional application No. 62/329,921, filed on Apr. 29, 2016.
(60) Provisional application No. 62/329,887, filed on Apr. 29, 2016.

(51) Int. Cl. A61B 5/00; A61B 5/16; A61B 3/113

(52) U.S. Cl. CPC A61B 5/4064; A61B 3/113; A61B 5/162

(57) ABSTRACT

A test is administered using a device responsive to touch for measuring the saccadic eye movement of an individual that has suffered some sort of trauma (impact or shaking) to the head, and comparing this measurement (post-trauma measurement or post-trauma CQ score) with a previous measurement taken prior to the impact (baseline measurement or baseline CQ score). The comparison yields a value test performance value resulting in a conclusion that the individual may be suffering from a concussion or that the individual is not in a concussive state. The test's accuracy is enhanced by (1) performing periodic baseline readings which are updated as the individual advances with age, (2) the near real-time of the post-trauma measurement in relation to when the trauma occurred, and (3) the automatic determination and transmission of objective test results resulting from an electronic touch screen system which can be transmitted to a networked physician.

### CROSS-REFERENCE TO RELATED APPLICATIONS

The present application claims priority to U.S. Provisional Application Ser. No. 62/329,921, entitled “System and Method for Rapid Healthcare Access”, filed Apr. 29, 2016, and to U.S. Provisional Application Ser. No. 62/329,887, entitled “System and Method for Detecting Traumatic Injury”, filed Apr. 29, 2016, the entire disclosure of each of which are hereby incorporated by reference.

### FIELD OF THE INVENTION

The present invention generally relates to the detection of traumatic brain injury. More specifically, detection is effectuated by an interactive process and system engaged prior to and after trauma to the head.

### BACKGROUND

A concussion is a type of traumatic brain injury caused by a bump, blow, jolt or other impact or vibration to the head that can change the way the brain normally functions. Symptoms usually reflect a functional disturbance to the brain, and may include physical (such as, headaches, nausea), cognitive (such as, difficulty with concentration or memory), emotional (such as, irritability, sadness), and other miscellaneous (such as, sleep disturbances, changes in appetite or energy levels) symptoms.

Each year, millions of concussions resulting from youth sports are reported each year. When factoring in concussions of professional sports as well as unreported incidents, the total number of concussions each year is even greater.

Because a concussion is a form of brain injury that requires immediate attention, it is important that this condition is identified with a high level of precision and in a timely manner, to allow for the administration of the appropriate recovery regiment.

### SUMMARY OF THE INVENTION

It is therefore essential to adopt a process and system that allows for quick screening for a risk of concussion with a high degree of accuracy.

In accordance with an embodiment of the invention, a test is administered using a device responsive to touch for measuring the saccadic eye movement of an individual that has suffered some sort of trauma (impact or shaking) to the head, and comparing this measurement (post-trauma measurement) with a previous measurement taken prior to the impact (baseline measurement). The comparison yields a value test performance value resulting in a meaningful deviation from the baseline measurement or that the individual is not in a concussive state.

The test's accuracy is enhanced by (1) performing periodic baseline readings which are updated as the individual advances with age, (2) the near real-time of the post-trauma measurement in relation to when the trauma occurred, and (3) the automatic reading and transmission of objective test results resulting from, for example, an electronic touch screen system, (4) the implementation of a logic measurement tool used in conjunction with the test results described in (3) above, and (5) evaluating the individual's postural stability (also referred to as balance).

These and other aspects of the invention will be more fully described below.

### BRIEF DESCRIPTION OF THE DRAWINGS

FIG. 1 is a screenshot of a level one saccadic measurement tool, in accordance with an embodiment of the invention.

FIG. 2 is a screenshot of a level one logic measurement tool, in accordance with an embodiment of the invention.

FIG. 3 is a screenshot of a level two saccadic measurement tool, in accordance with an embodiment of the invention.

FIG. 4 is a screenshot of a level three saccadic measurement tool, in accordance with an embodiment of the invention.

FIG. 5 is an illustration of a handheld mobile device displaying the screenshot of FIG. 3.

FIG. 6 is a schematic illustrating communications and transfer of data among a user mobile device, a physician network and payment clearinghouse, in accordance with an embodiment of the invention.

FIG. 7 is flowchart illustrating an exemplary process for implementing the evaluation tool shown in FIGS. 1-5 using the network shown in FIG. 6, in accordance with an embodiment of the invention.

### DETAILED DESCRIPTION OF THE INVENTION

While certain conventional concussion evaluation tools entail, for example, an evaluation of a patient's responsiveness post-trauma as compared with a pre-trauma (baseline) evaluation, these techniques carry certain aspects of unreliability. A major cause of unreliability results from the form of evaluations provided (for example, simple question and answers to measure responsiveness) and the delay that transpires between (i) when the impact occurs and when reliable examination is administered, and/or (ii) when the impact occurs and when a patient is examined by a trained physician.

The technique provided herein, in accordance with various embodiments of the invention, addresses these deficiencies. This is achieved by (i) implementing an evaluation tool that reliably determines whether brain function has been impacted by trauma to a patient's head, and (ii) establishing a communications network and process that effectively connects a patient, test administrator, physician and payment clearinghouse, along with pertinent information and permissions, to allow for timely assessment of the patient's condition.

It should be noted that, in this description, the term “user”, “individual tested”, and “athlete” are used interchangeably.

### 1. Concussion Evaluation Tool

The concussion evaluation tool described herein entails two comparative measurements—a baseline measurement (also referred to as a “baseline concussive quotient score” or “baseline CQ score”) and a post-traumatic measurement (also referred to as a “post-traumatic concussive quotient score” or “post-traumatic CQ score”). These measurements utilize detecting the saccadic eye movement of an individual before and after trauma and comparing the differences between the two measurements. It also involves requiring the individual being tested to interact with a screen that detects touch for the recording of entries made by such individual.

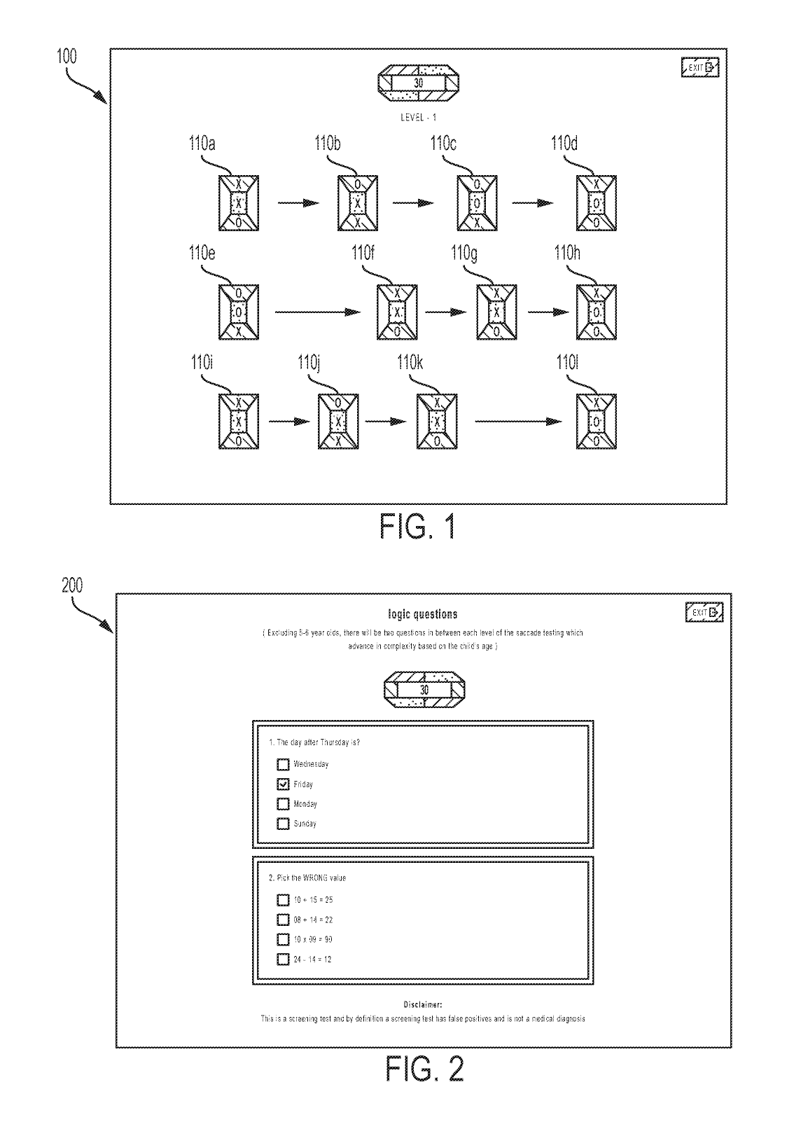

FIG. 1 shows a display 100 of a level one saccadic measurement tool, in accordance with an embodiment of the invention. Level one simply means that the intensity of the test is on the lower end of complexity of the testing spectrum; this would typically be used for young children. In this embodiment, the display 100 presents twelve icons 110a-1101. The individual being tested is instructed to start in the top left hand corner and then, following the arrows, move across from left to right. The individual is instructed to observe the character that is placed in the center (blue portion) of the first icon 110a (the character X) and to then touch the screen on that same icon 110a where the same character is recited again (the character X positioned in yellow). The individual is instructed to then continue on with the same exercise by moving across the top line (engaging icons 110b, 110c, 110d), then move to the second line from left to right (engaging 110e, 110f, 110g, 110h), followed by the third line (engaging 110i, 110j, 110k, 110l). This exercise will allow an individual's eye saccadic movements to be measured. The time taken to complete the exercise as well as the accuracy in terms of correcting placement by touch, will factor into a baseline CQ score for the individual.

In accordance with an embodiment of the invention, it is expected that individuals being evaluated, will be exposed to a number of screens like the one shown in FIG. 1. In order to maintain the requisite user focus across multiple screens, a logic screen 200 (FIG. 2) is displayed in between each successive saccadic measurement screen. This screen will display a variety of age appropriate multiple choice questions as shown in FIG. 2 and will also require the individual being evaluated to touch the appropriate response. In accordance with one embodiment of the invention, if the individual cannot correctly answer the logic questions, the test is terminated and the individual may be advised to seek medical attention.

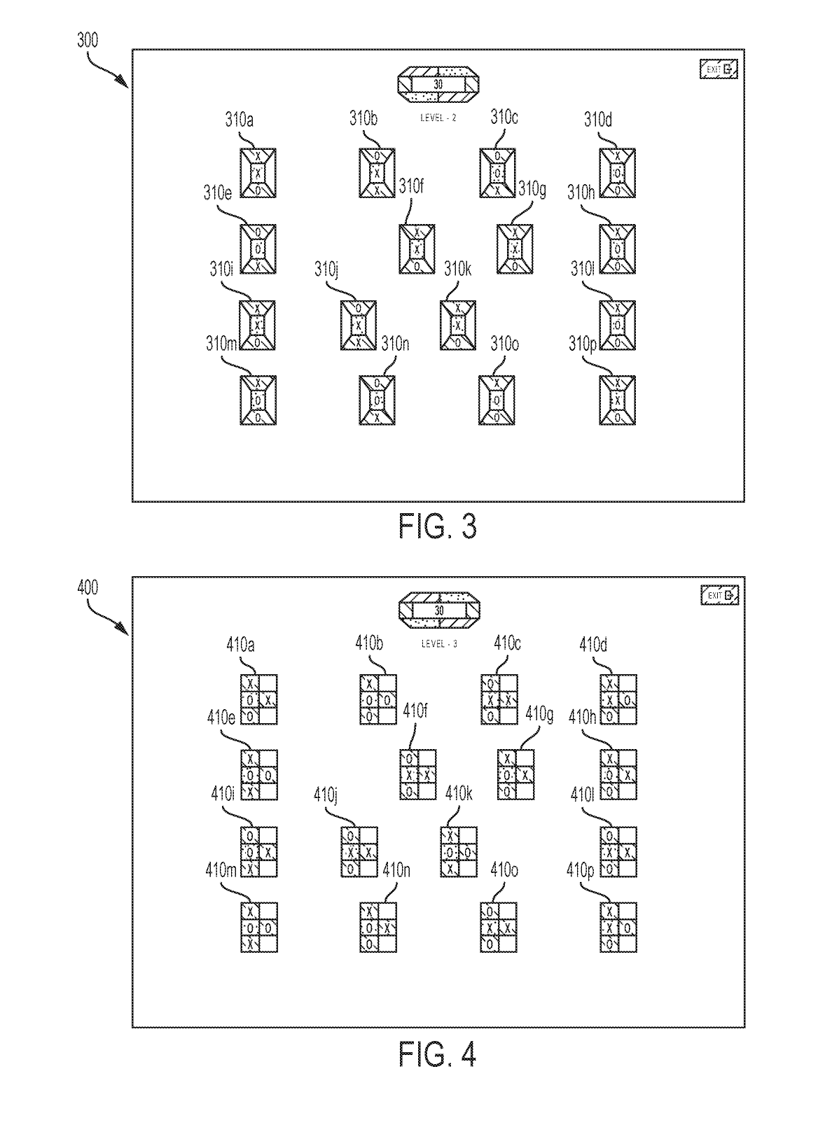

In accordance with another embodiment of the invention, individuals being evaluated are exposed to displays of icons that are laid out in a manner more complex than level 1. For example, in accordance with an embodiment of the invention, FIGS. 3 and 4 show level 2 (300) and level 3 (400) displays, respectively. The icons in the level 2 display —310a through 310p—are greater in number and are arranged less evenly on the screen. There are also no arrows that would serve as a reminder to the individual being tested as to the order of examination to be followed (i.e., left to right, starting at the top and heading from top to bottom). Turning to FIG. 4, the icons in the level 3 display—410a through 410p—have a further complexity as compared to what is show in FIG. 3, in that the level three icons have more touchscreen choices while only one correct answer remains.

The tools shown in FIGS. 1, 3 and 4 allow for the measurement of saccadic eye movement based on the number of correct and incorrect answers and the time taken to complete the page. It should be noted that, while statistical analyses of the average number of correct answers and the time for completion, serve as a useful understanding of the tool's effectiveness, the subjective baseline testing and subsequent post-trauma testing yield meaningful detection of a concussive condition or lack thereof.

As will be described below, the saccadic measurement tool will be used when the individual is not suffering from brain traumatic injury and, to complete the evaluation, after (ideally shortly or even better immediately after) trauma to the individual's head. This will allow for at least two measurements to be evaluated and, most significantly, any degenerative change in such measurement to be appreciated.

The systems and processes described thus far comprise two forms of evaluation: logic testing and saccadic eye movement measurement. In accordance with an embodiment of the invention, a third component is measured and factored into the individual's score; this component is postural stability (or balance). Upon impact, the individual's balance is measured—prior to or after the aforementioned logic and saccadic eye movement evaluations—and if the individual demonstrates postural instability, such finding is factored into the overall post-impact screening. Postural instability may be measured in a variety of ways. Three conventional techniques are: (i) the Balance Error Scoring System (BESS), (ii) Sensory Organization Test (SOT), and (iii) Balance Accelerometer Measure (BAM). The BESS entails three stances on two different surfaces—a firm surface and a foam surface—and requires the individual that is being screened to perform a double leg stance (i.e., the feet are flat on the testing surface approximately pelvic width apart), a single leg stance (i.e., the individual stands on the non-dominant leg with the contralateral limb held in approximately 20° of hip flexion, 45° of knee flexion and neutral position in the frontal plane) and a tandem stance (i.e., one foot is placed in front of the other with heel of the anterior foot touching the toe of the posterior foot). This evaluation may be performed using an application on a computer device (such as handheld device) or without the aid of an electronic device. The SOT identifies problems with postural control by assessing the patient's ability to make effective use of (or suppress inappropriate) visual, vestibular, and proprioceptive information. Because the SOT requires accessibility to certain equipment—such as a computer and force plates—this test may not be ideal for sideline screenings at a sporting event. BAM measures pelvic acceleration using a system that consists of a dual-axis accelerometer, a battery and a Bluetooth transmitter. Like the SOT, because the BAM requires the availability and use of advanced technology, BESS may be the preferred process for measuring postural instability. Nevertheless, any of these processes may be used. Detection postural stability or instability, as the case may be, of an individual being screened for concussion should be included as part of post-impact screening and factored into the individual's screening score; the results of such evaluation should be communicated to the physician that will be making the ultimate concussion diagnosis.

### 2. Communications Network

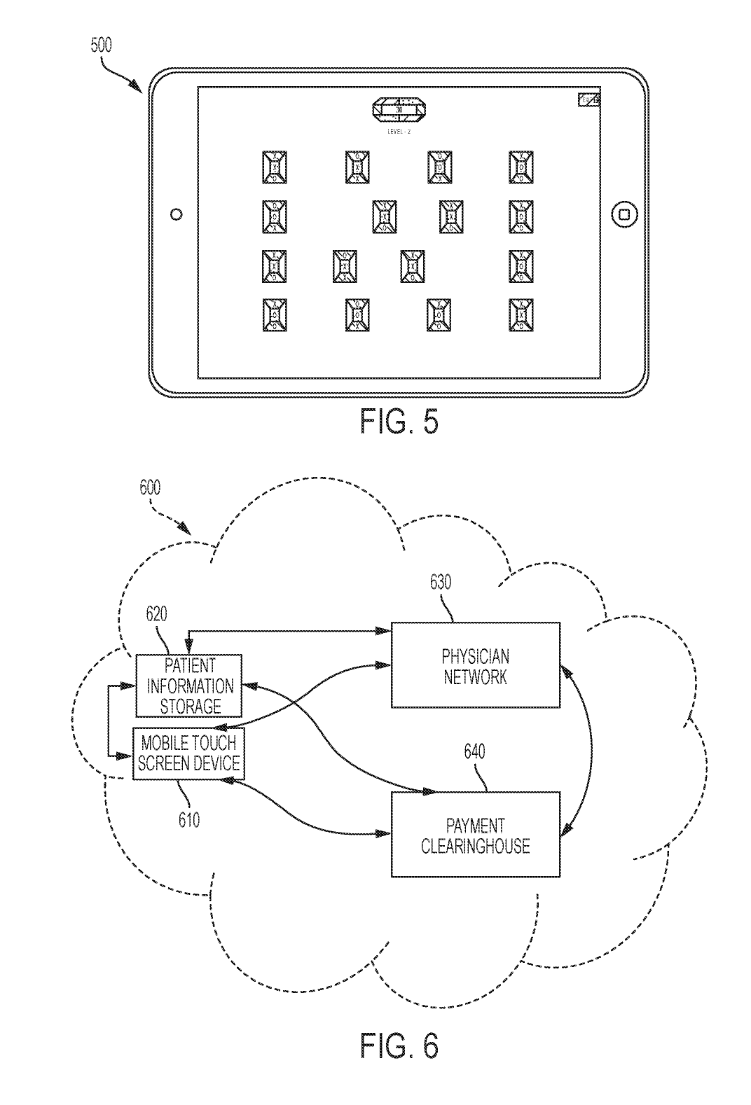

As described above, in accordance with an embodiment of the invention, it is anticipated that measurements using the concussion evaluation tools shown in FIGS. 1 through 4 are collected using a touch screen device, such as a tablet illustrated in FIG. 5. It may of course be appreciated that, other forms of response recordation may be used (such as voice (oral) responses) and with the use of other hardware (touchscreen computer, microphone), and the like.

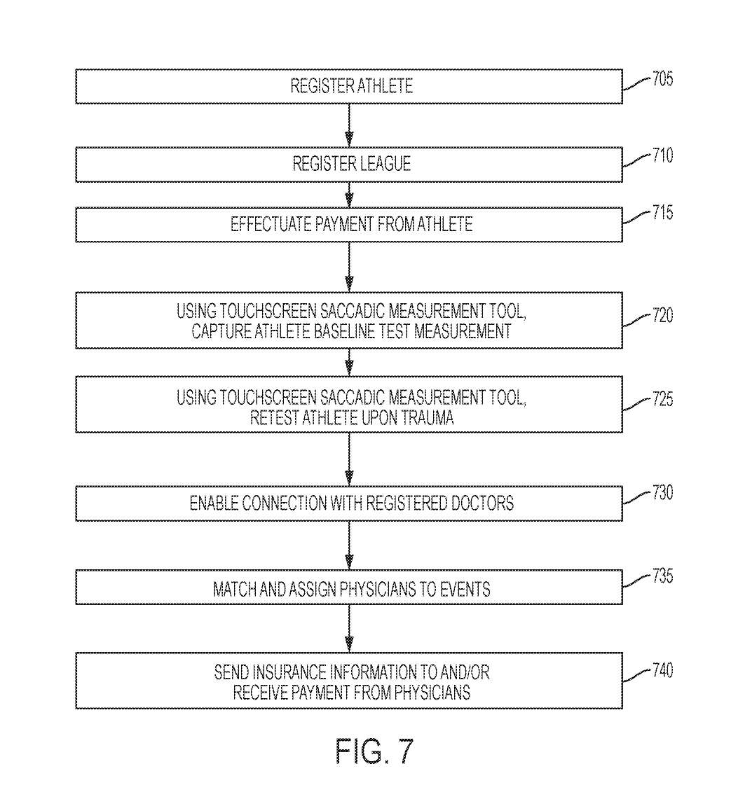

Turning to FIGS. 6 and 7, the system and process for effectuating saccadic evaluation for evaluating concussions, as well as connecting users with physicians, are described. The system and process also allows for the exchange of key information, including evaluation test scores, health insurance information, patient information, physician information, and payment information. More specifically, in accordance with an embodiment of the invention, FIG. 6 shows network 600 that supports such communication and data transmission. The network is envisaged to utilize some sort of wide area network (WAN) over the Internet, but it is appreciated that other systems may be used (such as local area network (LAN)) and other transmission systems (such as telephone (land and/or cellular)). In accordance with an embodiment of the invention, network 600 comprises mobile touch screen device 610, patient information storage 620, physician network 630 and payment clearinghouse 640.

As described above, the touch screen device 610 may be in the form of a tablet, laptop or desktop computer, or the like and is used to present the evaluation tools, receive user responses, transmit user information, communicate with a physician, and making payment for using the concussion evaluation tool and other related services (such as medical advice from a licensed physician through telemedicine).

Patient information storage 620 comprises data storage device(s) that store information relating to the user. This information typically would entail patient contact information, concussion evaluation test scores, payment information, and physician information.

Physician network 630 makes available, in the network 600, a number of physicians (ranging from a few local physicians to many throughout the country or abroad) that can evaluate a patient's trauma to the head by reviewing concussion evaluation scores, medical history and the like. These physicians can also provide medical advice over the network 600. Further, fund transfers—whether to the physician for services performed or by the physician for being part of the network 600—can be established.

Funds transfers, and data transfers related thereto, may in accordance with an embodiment of the invention, be effectuated through a payment clearinghouse 640. The payment clearing house would typically have access to banking information of a user, stored in mobile touch screen device 610 or patient information storage 620, as well as that of the network physicians stored in physician network 630.

Turning to FIG. 7, the process for evaluating whether an individual, such as an athlete, has suffered a concussion is illustrated in process flow steps 705 to 740.

First, as shown in step 705, an athlete is registered with the concussion evaluation network described above. In the youth sports setting, typically a parent will register a child by providing profile data (e.g., age, gender, activities, medical history). The member registration provides full access to evaluation for storage and comparison testing during games and throughout a given period of time (e.g., a year). By registering as a paid member, the athlete will receive evaluations, for example, for a given period of time and receive official dated scores that are stored whenever the member takes the evaluation, and will then be available for that member for comparison if an injury occurs during a game for take-out-of-play decision making. Athletes may register on their own or they may be asked to register by the league in which they participate (step 710). In accordance with an embodiment of the invention, the league may pay for the option of a having a team of remote physicians on call should one of its players suffer trauma to the head. In other embodiments, the fee is paid by the athlete or athlete's family (step 715).

In step 720, an athlete uses the concussion evaluation tool, such as tool 500 (show in FIG. 5), to establish a baseline. Test results are then saved in storage system 620 for subsequent availability to the athlete, athlete's parents or guardian and/or physicians. In accordance with an embodiment of the invention, the athlete may take the baseline (pre-trauma) evaluation only once in a given period of time or multiple times.

Then, an athlete may use the concussion evaluation tool, upon an impact to the head (step 725). At that point in time, by accessing patient information from storage device 620—including the athlete's baseline CQ score—a comparison between the baseline CQ score and the post-traumatic CQ score may be generated.

In step 730, physicians register such that they are available to participate in the overall concussion evaluation network 600. The physician's profile information is received and stored in physician network 630. This information includes, among other things, payment method (stored credit card information) to be used when the doctor responds to an injury incident at an athletic event.

A league's team coach can input its player data and, working with the league, can schedule individual player's test. Physician pool is identified for event during event registration process.

Should a registered athlete suffer a trauma to the head, in addition to utilizing the concussion evaluation tools described above the athlete is assigned a physician that is participating in physician network 630. This is made possible by making available a registry of network physicians and the physician is selected during the athletic event registration process. More specifically, in accordance with an embodiment of the invention, all scheduled league games, as they are input, shall be offered to physicians eligible in a given territory to ensure ongoing notice for interested physicians to participate by giving notice of available games. A physician that selects a game should be given priority to cover such game. Once, however, game selection priority is given to that physician, in order to maintain priority status as a covering physician, he or she must be responsive within a predetermined timeframe; otherwise, they shall lose the ability to cover the game so the second priority physician can be engaged. If there is an injury incident at an athletic event, a network physician is notified via email that services are required. At that time, the details of the incident are made available to the physician (injured person profile plus details of injury).

Also at this point, the physician's account (e.g., credit card) will be charged a network fee and the physician will receive insurance details to enable the doctor to bill the insurance company for services rendered at the athletic event. Physicians will be charged for using the network 600 when their services are required (injury incident is reports at an event). The network 600 will provide the physician with insurance information required for them to bill the insurance company for their services (step 740).

What is claimed is:

1. A method for evaluating a user for a traumatic brain injury, comprising:
initially subjecting the user to a computerized saccadic measurement tool, said tool encouraging (i) movement of the user's eyes horizontally across a screen for the tracking of icon sets in a predetermined order and (ii) selection of matching figures comprising each of said icon sets in the predetermined order;
initially recording a number of times the user successfully selects said matching figures within a predetermined period of time;
repeating said subjecting and recording steps, within a predetermined period of time after the user has suffered trauma to said users head; and
comparing performance between the repeated recording and the initial recording to yield evaluative indication information that the user may be in a concussive state.

2. The method of claim 1, wherein each recording yields a respective performance score and wherein said indication is determined by comparing a pre-trauma performance score and a post-trauma performance score.

3. The method of claim 2, wherein the selection is effectuated by touching a computerized screen.

4. The method of claim 1 wherein the user is subjected to logic testing after the user has been subjected to head trauma and prior to post-trauma recording.

5. The method claim 2, wherein the performance scores are based, in part, on the duration of time to effectuate said matching selections.

6. The method of claim 1, wherein the matching figure selections are arranged in a more complex manner, said complexity based on user age.

7. The method of claim 4, wherein a pre-trauma postural stability evaluation and a post-trauma postural evaluation are performed, and said postural stability evaluations are also utilized for yielding said evaluative indication information that the user may be in a concussive state.

8. A system for evaluating a user for a traumatic brain injury by measuring for the saccadic movement of the user's eyes, comprising:
a display of icons to encourage (i) movement of the user's eyes horizontally across a screen for the tracking of icon sets in a predetermined order and (ii) selection of matching figures comprising each of said icon sets in the predetermined order;
storage for initially recording a number of times the user successfully selects said matching figures within a predetermined period of time; and
a processor for comparing said movement and selection prior to a traumatic brain injury event and after said traumatic brain injury event, wherein said comparison yields evaluative indication information that the user may be in a concussive state.

9. The system of claim 8, wherein said comparison by the processor yields a respective performance score and wherein said indication information is determined by comparing a pre-trauma performance score and a post-trauma performance score.

10. The system of claim 9, wherein the selection is effectuated by touching a computerized screen.

11. The system of claim 9, wherein the processor enables said scores to be transmitted over a communications network.
